(19) United States
(12) Patent Application Publication
(10) Pub. No.: US 2005/0214933 A1
(43) Pub. Date: Sep. 29, 2005

(54) Ultrasonic micromixer with radiation perpendicular to mixing interface

(75) Inventors: Pil Woo Heo, Daejon (KR); Deok Jong Kim, Daejon (KR); Jae Yun Kim, Daejon (KR); Sang Jin Park, Daejon (KR); Eui Soo Yun, Daejon (KR)

Correspondence Address: BOYLE FREDRICKSON NEWHOLM STEIN & GRATZ, S.C., 250 E. WISCONSIN AVENUE, SUITE 1030, MILWAUKEE, WI, 53202, US

(73) Assignee: Korea Institute of Machinery & Materials

(21) Appl. No.: 10/993,540

(22) Filed: Nov. 19, 2004

(30) Foreign Application Priority Data
Dec. 19, 2003 (KR) 10-2003-0093895

(51) Int. Cl. G01N 33/569; G01N 33/53; C12M 1/34

(52) U.S. Cl. 435/287.2; 366/108

(57) ABSTRACT

The present invention provides an ultrasonic micromixer which is used in fields of bio-MEMS (micro-electro-mechanical-system) and μ-TAS (total analysis system) and radiates ultrasonic waves using a piezoelectric device in a direction perpendicular to a horizontal mixing interface between a plurality of sample fluids flowing in a chamber, so as to accelerate the mixing of the sample fluids, thus producing an evenly mixed sample. The ultrasonic micromixer includes a plurality of independent inlets, and a plurality of guide channels which are coupled to the inlets at different heights. The ultrasonic micromixer further includes the chamber to allow the plurality of sample fluids, discharged from the guide channels, to mix with each other while flowing through the chamber, and an outlet to discharge the mixed sample fluids. The ultrasonic micromixer further includes a vibration plate which is attached to a lower portion of the chamber, and a piezoelectric device which is attached to a lower surface of the vibration plate to radiate ultrasonic waves in a vertical direction. Therefore, the ultrasonic micromixer achieves miniaturization, accuracy and integration of devices used in the fields of the bio-MEMS and μ-TAS.

### BACKGROUND OF THE INVENTION

1. Field of the Invention

The present invention relates generally to ultrasonic micromixers which are used in fields of bio-MEMS (micro-electro-mechanical-system) and μ-TAS (total analysis system) to mix a plurality of sample fluids passing through micro channels, and more particularly, to an ultrasonic micromixer which provides a horizontal mixing interface between a plurality of sample fluids flowing in a chamber and radiates ultrasonic waves using a piezoelectric device in a direction perpendicular to the horizontal mixing interface between the sample fluids, thus enhancing the mixing efficiency of the sample fluids, thereby producing an evenly mixed sample.

2. Description of the Related Art

Recently, bio-diagnosis techniques have been developed for miniaturization, accuracy and integration of devices used in the bio-diagnosis fields. Miniaturization of the devices provides several advantages, as follows: first, the amount of expensive biomass sample used in bio-diagnosis is reduced. Second, a reduction in circumferential noise enhances the sensitivity of the devices used in the bio-diagnosis fields. Third, several biomass samples can be treated in parallel, thus reducing processing time.

However, sample fluids such as biomass pass through micro channels. Therefore, issues which are unimportant in channels of macro sizes may arise.

In a detailed description, a plurality of sample fluids passing through the micro channels has a lower Reynolds number (Re<<2000). Thus, the sample fluids do not have turbulence flows, but generate laminar flows in the micro channels. Accordingly, the sample fluids flowing in the micro channels are mixed only by diffusion.

In the micro channels having the above-mentioned properties, to evenly mix the plurality of sample fluids, sufficient processing time is required for diffusion of the sample fluids. Therefore, the micro channels must have sufficient lengths. As such, due to the long micro channels, the miniaturization of the devices used in the bio-diagnosis fields is very difficult.

FIG. 1 is a sectional view showing a conventional ultrasonic micromixer. In the conventional ultrasonic micromixer, two sample fluids are drawn into a chamber 40 through first and second inlets 10 and 20 which are provided on an upper surface of the chamber 40. Thereafter, the two sample fluids are mixed with each other while flowing through the chamber 40, prior to being discharged through an outlet 30.

In the conventional ultrasonic micromixer having the above-mentioned structure, a mixing interface between the two sample fluids is vertical while the two sample fluids flow through the chamber 40. A vibration plate 60 and a piezoelectric device 50 are attached to a lower surface of the chamber 40. The vibration plate 60 and the piezoelectric device 50 vertically radiate ultrasonic waves to mix the two sample fluids. However, because the radiation direction of the ultrasonic waves is parallel to the mixing interface between the two sample fluids, the effect of the ultrasonic waves is deteriorated.

### SUMMARY OF THE INVENTION

Accordingly, the present invention has been made keeping in mind the above problems occurring in the prior art, and an object of the present invention is to provide an ultrasonic micromixer which includes flowing channels having improved structure such that a mixing interface between a plurality of sample fluids is horizontal while the sample fluids flow in a chamber, thus enhancing mixing efficiency of the sample fluids, and which has a piezoelectric device to radiate ultrasonic waves in a direction perpendicular to the horizontal mixing interface between the sample fluids, thus accelerating the mixing of the sample fluids.

In order to accomplish the above object, the present invention provides an ultrasonic micromixer with radiation perpendicular to the mixing interface to mix a plurality of sample fluids passing through micro channels. The ultrasonic micromixer includes a plurality of independent inlets; a plurality of guide channels coupled to the inlets at different heights; a chamber to allow the plurality of sample fluids, discharged from the guide channels, to be mixed with each other while flowing through the chamber; and an outlet to discharge the mixed sample fluids.

The ultrasonic micromixer may further include a vibration plate attached to a lower portion of the chamber, and a piezoelectric device attached to a lower surface of the vibration plate to radiate ultrasonic waves in a vertical direction.

Therefore, if reliable operation is required, the active ultrasonic micromixer using the piezoelectric device is used in this field. The active ultrasonic micromixer is a device to enhance the mixing efficiency of the sample fluids using an outside energy source. The active ultrasonic micromixer radiates ultrasonic waves to cause the sample fluids to generate a turbulent flow in the chamber of the active ultrasonic micromixer, thus easily mixing the sample fluids. The active ultrasonic micromixer is insensitive to gas bubbles and is capable of controlling the mixing of the sample fluids according to the level of input energy.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a sectional view showing a conventional ultrasonic micromixer;

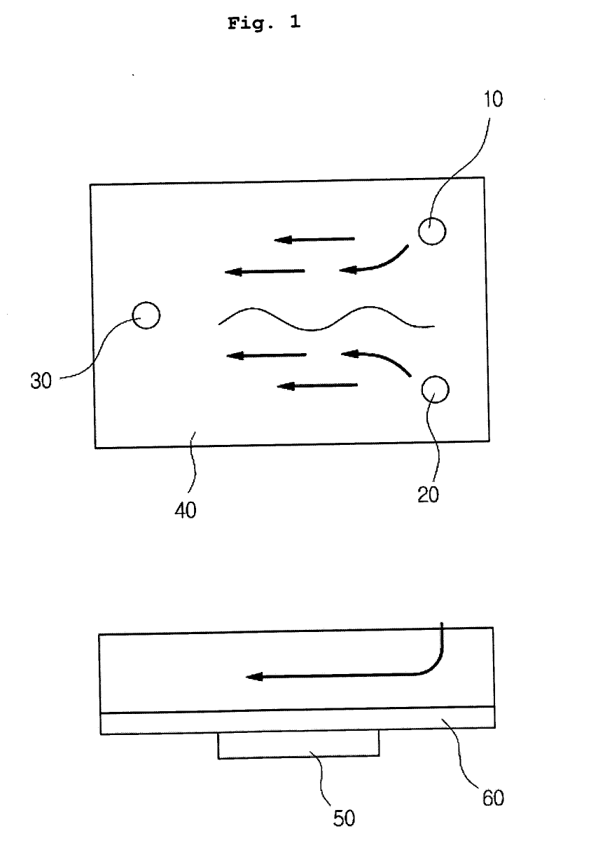

FIG. 2 is a plan view of an ultrasonic micromixer with radiation perpendicular to a mixing interface, according to a preferred embodiment of the present invention; and

FIG. 3 is a sectional view of the ultrasonic micromixer of FIG. 2.

### DESCRIPTION OF THE PREFERRED EMBODIMENTS

Hereinafter, a preferred embodiment of the present invention will be described in detail with reference to the attached drawings.

Reference now should be made to the drawings, in which the same reference numerals are used throughout the different drawings to designate the same or similar components.

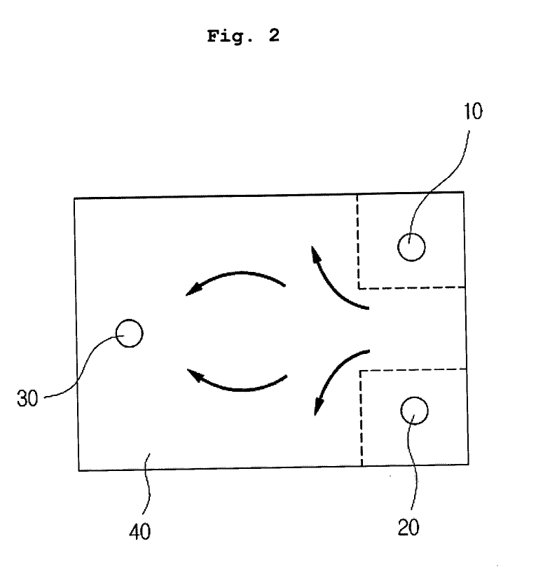

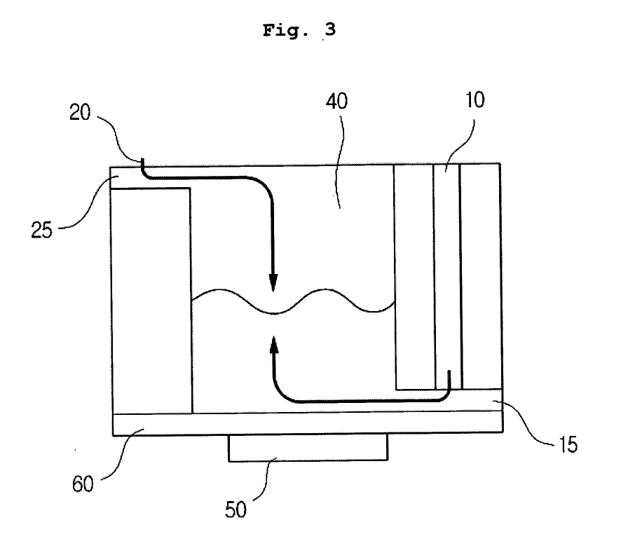

FIG. 2 is a plan view of an ultrasonic micromixer with radiation perpendicular to a mixing interface, in which first and second inlets 10 and 20 and an outlet 30 are provided on an upper surface of a chamber 40, according to a preferred embodiment of the present invention. FIG. 3 is a sectional view of the ultrasonic micromixer of FIG. 2 to show the first and second inlets 10 and 20, first and second guide channels 15 and 25 and a chamber 40.

Referring to FIGS. 2 and 3, the ultrasonic micromixer according to the preferred embodiment of the present invention mixes a plurality of sample fluids passing through micro channels. The ultrasonic micromixer includes the first and second inlets 10 and 20 which are provided on an upper surface of the chamber 40, independently. The sample fluids are injected into the chamber 40 through the first and second inlets 10 and 20. The ultrasonic micromixer further includes the first and second guide channels 15 and 25 which are independently coupled to the first and second inlets 10 and 20, respectively.

Preferably, the first and second guide channels 15 and 25 are provided at different heights in the chamber 40. Thus, the plurality of sample fluids passing through the first and second inlets 10 and 20 are drawn at different heights into the chamber 40 via the first and second guide channels 15 and 25. In an early stage of inflow of the plurality of sample fluids, the sample fluids are vertically layered in the chamber 40.

Therefore, the ultrasonic micromixer of the present invention, in which the sample fluids are mixed with each other while being layered on top of one another in the chamber, has a mixing efficiency superior to conventional ultrasonic micromixers in which a plurality of sample fluids are mixed with each other while being divided vertically.

The ultrasonic micromixer according to the preferred embodiment of the present invention further includes a vibration plate 60 and a piezoelectric device 50 which are attached to a lower surface of the chamber 40, thus enhancing the efficiency of mixing the sample fluids flowing in the chamber 40 while being layered on top of another. In detail, the vibration plate 60 and the piezoelectric device 50 radiate vertical ultrasonic waves to the horizontal mixing interface between the sample fluids. The above-mentioned ultrasonic waves agitate the horizontal mixing interface between the sample fluids, thus accelerating the mixing of the sample fluids.

The process of mixing the sample fluids using the ultrasonic micromixer of the present invention will be described herein below.

The process of mixing the sample fluids using the ultrasonic micromixer of the present invention includes a step of injecting the sample fluids into a chamber 40 with the independent first and second inlets 10 and 20. The process of mixing the sample fluids further includes a step of outflow of the sample fluids through the first and second guide channels 15 and 25 which are provided at different heights. The process of mixing the sample fluids further includes a step of mixing the sample fluids, which are discharged through the first and second guide channels 15 and 25, in the chamber 40. The process of mixing the sample fluids further includes a step of vertically radiating ultrasonic waves from the piezoelectric device 50 attached to the lower portion of the chamber 40, and a step of vertical vibration of a vibration plate 60, which is attached to the lower portion of the chamber 40, by the ultrasonic waves radiated from the piezoelectric device 50. The process of mixing the sample fluids further includes a step of evenly mixing the sample fluids in the chamber 40 by the vertical vibration of the vibration plate 50, and a step of discharging the mixed sample fluids through the outlet 30 of the chamber 40.

To increase the mixing efficiency between the sample fluids which are mixed in the chamber 40 after the outflows through the first and second guide channels 15 and 25, when the sample fluids are injected into the first and second inlets 10 and 20, a sample fluid having a higher specific weight is injected into the second inlet 20 provided at a higher position. Then, the sample fluid having the higher specific weight flows downward, in response to gravity. Therefore, the sample fluid having the higher specific weight is efficiently mixed with a sample fluid having a lower specific weight.

As described above, the present invention provides an ultrasonic micromixer which radiates ultrasonic waves using a piezoelectric device in a direction perpendicular to a horizontal mixing interface between a plurality of sample fluids flowing in a chamber, so as to enhance the mixing efficiency of the sample fluids, thus producing an evenly mixed sample, thereby achieving miniaturization, accuracy and integration of devices in the fields of bio-MEMS and μ-TAS.

Although the preferred embodiment of the present invention has been disclosed for illustrative purposes, those skilled in the art will appreciate that various modifications, additions and substitutions are possible, without departing from the scope and spirit of the invention as disclosed in the accompanying claims.

1. An ultrasonic micromixer with radiation perpendicular to a mixing interface, which mixes a plurality of sample fluids passing through micro channels, the ultrasonic micromixer comprising:
a plurality of independent inlets;
a plurality of guide channels coupled to the inlets at different heights;
a chamber to allow the plurality of sample fluids, discharged from the guide channels, to mix with each other while flowing through the chamber; and
an outlet to discharge the mixed sample fluids.

2. The ultrasonic micromixer according to claim 1, further comprising:
a vibration plate attached to a lower portion of the chamber; and
a piezoelectric device attached to a lower surface of the vibration plate to radiate ultrasonic waves in a vertical direction.

3. An ultrasonic micromixer with radiation perpendicular to a mixing interface, which mixes a plurality of sample fluids passing through micro channels, wherein a process of mixing the sample fluids comprises:
a step of injecting the sample fluids into a chamber with a plurality of independent inlets;
a step of outflows of the sample fluids through guide channels which are provided at different heights;
a step of mixing the sample fluids, discharged through the guide channels, in the chamber;
a step of vertically radiating ultrasonic waves from a piezoelectric device attached to a lower portion of the chamber;
a step of vertically vibrating a vibration plate, attached to the lower portion of the chamber, by the ultrasonic waves radiated from the piezoelectric device;
a step of evenly mixing the sample fluids in the chamber by the vertical vibration of the vibration plate; and
a step of discharging the mixed sample fluids through an outlet of the chamber.

4. The ultrasonic micromixer according to claim 3, wherein when the plurality of sample fluids is injected into the inlets, a sample fluid having a higher specific weight is injected into an inlet provided at a higher position in the chamber.
